(19) United States
(12) Patent Application Publication
(10) Pub. No.: US 2007/0104791 A1
(43) Pub. Date: May 10, 2007

(54) Method of preparing a pharmaceutical composition and pharmaceutical compositions obtainable thereby

(75) Inventors: Todor Alexandrov Popov, Sofia (BG); Christo Tzachev Tzachev, Sofia (BG)

Correspondence Address: GOMEZ INTERNATIONAL PATENT OFFICE, LLC, 1501 N. RODNEY STREET, SUITE 101, WILMINGTON, DE, 19806, US

(73) Assignee: Fortune Apex Development Limited, Tortola (VG)

(21) Appl. No.: 11/312,887

(22) Filed: Dec. 20, 2005

(30) Foreign Application Priority Data
Oct. 24, 2005 (GB) 0521708.8

(51) Int. Cl. A61K 9/14

(52) U.S. Cl. 424/488

(57) ABSTRACT

An aqueous pharmaceutical composition comprising: a) 0.005 to 10% by wt of one or more water-soluble pharmaceutically active ingredients or pharmaceutically acceptable salts thereof; b) from 0.01 to 10% by wt hydroxypropyl methyl cellulose having a viscosity of from 2500 to 5500 cps (mPa·s); and c) a buffer for maintaining the pH of the aqueous pharmaceutical composition from 5 to 7; can be prepared by a method which comprises: i) dissolving the above components in water to form an aqueous solution, and ii) filtering the aqueous solution formed in i) through a sieve having a mesh size from ≧1 micron but ≦10 microns. The compositions obtainable by this method are capable of demonstrating improved mucoadhesive consistency and stability.

This invention is concerned with a method of preparing a pharmaceutical composition and pharmaceutical compositions obtainable thereby. More particularly, this invention is concerned with a method of preparing an aqueous pharmaceutical composition which comprises one or more water soluble pharmaceutically active ingredients and hydroxypropyl methyl cellulose and to aqueous pharmaceutical compositions obtainable thereby.

Pharmaceutical compositions have been used for the treatment of rhinitis, sinusitis, hay fever and other inflammatory conditions of the nasal cavities for many years. Often, the pharmaceutical compositions are administered nasally in the form of drops or sprays. The success of treatment of a particular condition may not only depend upon the nature of the pharmaceutically active ingredient in pharmaceutical composition but can also depend upon the ability of the other ingredients in the composition to distribute and retain the active ingredient over the mucosal membranes within the nasal cavity and, if appropriate, the nasolacrimal canal. The distribution and retention properties of a pharmaceutical composition over a mucosal membrane are hereafter generally referred to as the mucoadhesive properties of the composition.

The mucoadhesive properties of a pharmaceutical composition are of great interest to pharmaceutical formulators and drug delivery scientists, as they can contribute to the controlled release of the active ingredient on the mucosal membranes.

The term “adhesion” refers to the intermolecular forces which hold matter together, particularly contiguous surfaces of neighbouring media. An “adhesive” is a substance capable of joining two materials by adhesion. “Bioadhesion” implies that at least one of the two materials is of biological origin. When one surface is the adherent mucus layer covering the mucosal epithelia, the term “mucoadhesion” is used. Mucoadhesion is specific type of bioadhesion, not a synonym thereof. Mucoadhesive materials may be useful to provide prolonged adhesion, and so improved efficacy, of pharmaceutically active ingredients on mucosal tissue.

Hydroxypropyl methyl cellulose (HPMC) is used in aqueous pharmaceutical compositions for the nasal administration of various pharmaceutically active ingredients. In these applications, the HPMC is being used to prolong the adhesion of the active ingredient on the mucosal membranes. Examples of prior art publications include U.S. Pat. No. 4,603,131, WO-A-03070213 and WO-A-99038492.

U.S. Pat. No. 4,603,131 discloses a composition for preventing and treating irritation of the mucous membranes of the nose, the composition comprising a tricyclic anti-depressant in combination with a vasoconstrictor. HPMC is disclosed as a possible viscosity agent. Other than describing the compositions of the worked examples as having been prepared at room temperature by conventional mixing techniques, this document provides no details of the preparation techniques employed.

WO-A-03-70213 discloses a liquid mucoadhesive pharmaceutical composition containing a pharmaceutically active substance e.g. xylometazoline hydrochloride in an amount of from 0.01 up to 10.00 wt %, mucoadhesive substance e.g. HPMC in an amount of from 0.1 up to 10 wt %, preservative e.g. disodium EDTA in an amount of from 0.01 up to 5.00 wt %, and a phosphate buffer system, and wherein the composition has a pH of from 5 to 7. In this document, a mucoadhesive solution is prepared by mixing HPMC and EDTA in small proportions to a warm phosphate buffer solution and allowing the solution to cool to 20° C. After the solution is allowed to stand for 24 hours and bubbles removed under reduced pressure, the active ingredient is added and stirred for 2 hours at 20° C. There is no disclosure in this document of the pharmaceutical composition being subjected to any filtering steps at any stage during its preparation.

WO-A-99038492 discloses an aqueous nasal pharmaceutical composition which comprises:
(a) one or more active substances suitable for nasal administration, such as vasoconstrictors, antiallergic agents and corticosteroids; and
(b) a water-soluble C₁-C₄ alkyl-cellulose derivative, such as HPMC.

In the preparation of the pharmaceutical composition, once all the ingredients have been mixed and dissolved, the final solution is disclosed to be filtered through a mesh screen of approximately 50 μm.

The HPMC-based pharmaceutical compositions prepared in accordance with the preparation processes disclosed in the above documents are difficult to formulate to a consistent level of mucoadhesion, particularly as the mucoadhesive properties of a specific composition can vary quite considerably from one temperature to another. Moreover, the known HPMC-based pharmaceutical compositions tend to demonstrate instability of mucoadhesion over an extended period of time.

It is known that bacteria, which generally have a size in the range of from 0.2 to 600 μm, can be removed from pharmaceutical preparations, or components thereof, to render them sterile by passing them through a sub-micron filter, for example as disclosed in US-A-2005058699.

It is an object of the present invention to produce aqueous pharmaceutical compositions to a consistent level of mucoadhesion.

It is a further object of the present invention to provide aqueous pharmaceutical compositions with improved mucoadhesive stability.

In accordance with a first aspect of the present invention there is provided a method for the preparation of an aqueous pharmaceutical composition comprising:
a) 0.005 to 10% by wt, preferably 0.01 to 5% by wt, more preferably 0.25 to 2.5 wt %, most preferably 0.25 to 1.5% by wt of one or more water-soluble pharmaceutically active ingredients or pharmaceutically acceptable salts thereof, such as a water-soluble pharmaceutically active ingredient or salt thereof selected from vasoconstrictors, antiallergic agents, antiemetics, bronchodilators, antiseptics, local anesthetics, cytostatics, analgesics (narcotic and non-narcotic), steroidal and non-steroidal anti-inflammatories, topical antibiotics, antiparasitics, antibacterials, anticonvulsants, antispasmodics and anticholinergics, antifungals, antivirals, antidiabetics, antimigraines, hormones, sedatives, antianaphylactics, beta-adrenoceptor agonists, diagnostic drugs, and vaccines;
   b) from 0.01 to 10% by wt, preferably from 0.05 to 5% by wt, more preferably 0.1 to 5% by wt, most preferably 0.1 to 2% by wt, hydroxypropyl methyl cellulose, having a viscosity of from 2500 to 5500 cps (mPa·s), preferably more than 3000 to less than 5000 cps (mPa·s), more preferably from 3200 to 4800 cps (mPa·s), (Ubbelohde, 2 wt % solution in water, 20° C., in accordance with United States Pharmacopoeia, hereafter “USP”), and c) a buffer for maintaining the pH of the aqueous pharmaceutical composition from 5 to 7, wherein the method comprises:
i) dissolving the above components in water to form an aqueous solution, and
ii) filtering the aqueous solution formed in i) through a sieve to form an aqueous pharmaceutical composition;
   characterised in that the sieve through which the aqueous solution is filtered has a mesh size from ≧1 micron but ≦10 microns, preferably from 1.5 to 5 microns, most preferably 2 to 4 microns, e.g. 3 microns.

In accordance with another aspect of the present invention there is provided an aqueous pharmaceutical composition comprising:
a) 0.005 to 10% by wt, preferably 0.01 to 5% by wt, more preferably 0.25 to 2.5 wt %, most preferably 0.25 to 1.5% by wt of one or more water-soluble pharmaceutically active ingredient or pharmaceutically acceptable salt thereof suitable for nasal administration, such as one or more water soluble pharmaceutically active ingredients selected from vasoconstrictors, antiallergic agents, antiemetics, bronchodilators, antiseptics, local anesthetics, cytostatics, analgesics (narcotic and non-narcotic), steroidal and non-steroidal anti-inflammatories, topical antibiotics, antiparasitics, antibacterials, anticonvulsants, antispasmodics and anticholinergics, antifungals, antivirals, antidiabetics, antimigraines, hormones, sedatives, antianaphylactics, beta-adrenoceptor agonists, diagnostic drugs, and vaccines;
b) from 0.01 to 10% by wt, preferably from 0.05 to 5% by wt, more preferably 0.1 to 5% by wt, most preferably 0.1 to 2% by wt, hydroxypropyl methyl cellulose, having a viscosity of from 2500 to 5500 cps (mPa·s), preferably more than 3000 to less than 5000 cps (mPa·s), more preferably from 3200 to 4800 cps (mPa·s), (Ubbelohde, 2 wt % solution in water, 20° C., in accordance with USP), and
c) a buffer for maintaining the pH of the aqueous pharmaceutical composition from 5 to 7,
   wherein the composition is obtainable or obtained by the method of the first aspect of the invention.

The pharmaceutical compositions made by the process of the present invention are solutions, thereby rendering them suitable for administration to mucosal epithelia. Typically, the compositions are for oral e.g. inhalatory, sublingual or peridontal, ocular, nasal, rectal, or vaginal administration. The compositions should be presented in a format suitable for appropriate administration, as would be understood by a person skilled in the art. For example, the composition may be administered as one or more drops (e.g. for nasal, ocular or oral administration), as a spray (e.g. for nasal or oral inhalatory administration), as an injectable liquid (e.g. for oral, rectal or vaginal administration), or as a mouthwash or thin syrup (for oral administration).

Whilst a person skilled in the art would expect aqueous pharmaceutical compositions made with HPMC to demonstrate excellent mucoadhesive properties, it has been found that the compositions made by the process of the first aspect of the present invention demonstrate surprisingly consistent mucoadhesive properties in comparison to similarly formulated compositions which are either not filtered or are filtered through a sieve having a mesh size significantly greater than 10 microns. Further, the compositions of the present invention retain their mucoadhesive properties for surprisingly longer than similarly formulated compositions which have not been filtered or which have been filtered with larger mesh sizes.

It is believed that the above benefits of the present invention will, to a greater or lesser extent, be demonstrated for all water soluble pharmaceutically active ingredients. However, it is preferred that the pharmaceutically active ingredients employed in the present invention are water soluble vasoconstrictors, antiallergic agents, antiemetics, bronchodilators, antiseptics, local anesthetics, cytostatics, analgesics (narcotic and non-narcotic), steroidal and non-steroidal anti-inflammatories, topical antibiotics, antiparasitics, antibacterials, anticonvulsants, antispasmodics and anticholinergics, antifungals, antivirals, antidiabetics, antimigraines, hormones, sedatives, antianaphylactics, beta-adrenoceptor agonists, diagnostic drugs, and vaccines.

In the present invention, suitable water-soluble vasoconstrictors may be selected from xylometazoline e.g. xylometazoline hydrochloride, xylometazoline, indanazoline, metizoline, naphazoline e.g. naphazoline hydrochloride, fenoxazoline e.g. fenoxazoline hydrochloride, oxymetazoline e.g. oxymetazoline hydrochloride, tetrahydrozoline, tramazoline, tymazoline, phenylephrine e.g. phenylephrine hydrochloride, ephedrine e.g. d-pseudoephedrine hydrochloride, or epinephrine. Preferably, the water-soluble active ingredient is selected from xylometazoline, e.g. xylometazoline hydrochloride, and oxymetazoline, e.g. oxymetazoline hydrochloride. In one embodiment of this invention, the water-soluble active ingredient is xylometazoline, e.g. xylometazoline hydrochloride.

In the present invention, suitable water soluble antiallergic agents may be selected from (1) cromoglycic acid or a pharmaceutically acceptable salt thereof, e.g. disodium cromoglycate), or (2) H1 receptor antagonists, such as dimethindene or a pharmaceutically acceptable salt thereof, e.g. dimethindene maleate, acrivastine, azelastine, brompheniramine, chlorpheniramine, dexchlorpheniramine, triprolidine, bromodiphenhydramine, clemastine, phenyltoloxamine, piprinhydrinate, pyrilamine, tripelennamine, cetirizine, levocetirizine, hydroxyzine, methdilazine, promethazine, trimeprazine, azatadine, cyproheptadine, loratadine, desloratadine, astemizole, diphenhydramine, or levocabastine orterfenadine.

In the present invention, suitable water soluble anti-inflammatories include the steroidal anti-inflammatories e.g. corticosteroids, such as those corticosteroids selected from beclomethasone e.g. beclomethasone dipropionate, and fluticasone e.g. fluticasone propionate, and non-steroidal anti-inflammatories e.g. diclofenac and celecoxib.

In the present invention, suitable water soluble antiemetics may be selected from metoclopramide such as metoclopramide hydrochloride, ondansetron, granisetron, dronabinol, prochloperazine and chlorpromazine. In one embodiment of this invention, the water-soluble active ingredient is metoclopramide such as metoclopramide hydrochloride.

In the present invention, suitable water-soluble narcotic and non-narcotic analgesics include e.g. morphine, hydromorphine, pentazocine, and acetaminophen.

In the present invention, suitable water-soluble anesthetics include local anesthetics such as lidocaine, pramoxine, and benzocaine.

In the present invention, suitable water-soluble topical antibiotics include neomycin and bacitracin.

In the present invention, suitable water-soluble antiparasitics include metronidazole and quinolines.

In the present invention, suitable water-soluble antibacterials include tetracycline, erythromycin, quinolone antibacterials, and azithromycin.

In the present invention, suitable water-soluble anticonvulsants include phenyloin, gabapentin, phenobarbital and carbamazepine.

In the present invention, suitable water-soluble antispasmodics and anticholinergics include atropine and scopolamine.

In the present invention, suitable water-soluble antifungals include miconazole, econazole and terconazole.

In the present invention, suitable water-soluble antivirals include acyclovir and behenyl alcohol.

In the present invention, suitable water-soluble antidiabetics include glipizide and glyburide.

In the present invention, suitable water-soluble antimigraines include sumatriptan and ergotamine.

In the present invention, suitable water-soluble hormones include insulin, steroidal hormones, calcitonin, melatonin and tissue growth factors.

In the present invention, suitable water-soluble sedatives include barbiturates and benzodiazepines.

In the present invention, suitable water-soluble antianaphylactics include adrenaline and epinephrine.

In the present invention, suitable water-soluble beta-adrenoceptor agonists include ephedrine Hydrochloride, salbutamol/albuterol, fenoterol, clenbuterol, salmeterol and formoterol.

In the present invention, suitable water-soluble diagnostic drugs include phenolsulfonphthalein, Dye T-1824, vital dyes, potassium ferrocyanide, secretin, pentagastrin and cerulean.

In the present invention, suitable water-soluble vaccines include allergens for immunotherapy and oral bacterial vaccines used as immunomodulators.

In the present invention, all the water soluble active ingredients which are capable of salt formation may be present in the pharmaceutical composition either in free form or in the form of a pharmaceutically acceptable salt.

In the present invention, mixtures of more than one water soluble active ingredient may be employed in the pharmaceutical composition, e.g. a combination of a vasoconstrictor and an antiallergic agent, such as xylometazoline plus cromoglycic acid or phenylephrine plus dimethindene, or a combination of a vasoconstrictor and a corticosteroid, such as xylometazoline plus beclomethasone.

In the present invention, the water-soluble pharmaceutically active ingredient is preferably selected from one or more of xylometazoline, indanazoline, metizoline, naphazoline, fenoxazoline, oxymetazoline, tetrahydrozoline, tramazoline, tymazoline, phenylephrine, ephedrine, epinephrine, cromoglycic acid, dimethindene, acrivastine, azelastine, brompheniramine, chlorpheniramine, dexchlorpheniramine, triprolidine, bromodiphenhydramine, clemastine, phenyltoloxamine, piprinhydrinate, pyrilamine, tripelennamine, cetirizine, levocetirizine, hydroxyzine, methdilazine, promethazine, trimeprazine, azatadine, cyproheptadine, loratadine, desloratadine, astemizole, diphenhydramine, levocabastine, terfenadine, beclomethasone, fluticasone, diclofenac, celecoxib, metoclopramide, ondansetron, granisetron, dronabinol, prochloperazine, chlorpromazine, morphine, hydromorphine, pentazocine, acetaminophen, lidocaine, pramoxine, benzocaine, neomycin, bacitracin, metronidazole, tetracycline, erythromycin, quinolone antibacterials, azithromycin, phenyloin, gabapentin, phenobarbital, carbamazepine, atropine, scopolamine, miconazole, econazole, terconazole, acyclovir, behenyl alcohol, glipizide, glyburide, sumatriptan, ergotamine, insulin, steroidal hormones, calcitonin, melatonin, tissue growth factors, barbiturates, benzodiazepines, adrenaline, epinephrine, salbutamol/albuterol, fenoterol, clenbuterol, salmeterol, formoterol, phenolsulfonphthalein, Dye T-1824, vital dyes, potassium ferrocyanide, secretin, pentagastrin, cerulean, allergens for immunotherapy and oral bacterial vaccines used as immunomodulators, or a pharmaceutically acceptable salt thereof.

The hydroxypropyl methyl cellulose (HPMC) employed in the present invention must be of a grade which itself has a viscosity of from 2500 to 5500 cps (mPa·s). Preferably, the HPMC has a viscosity of more than 3000 to less than 5000 cps (mPa·s), more preferably from 3200 to 4800 cps (mPa·s), e.g. 4000 cps (mPa·s). Viscosity of the HMPC is measured using an Ubbelohde viscometer on a 2 wt % solution in water at 20° C., in accordance with USP. Suitable grades of HPMC materials which achieve the specified viscosities are well know to those skilled in art and are commercially available from various sources such Dow, Hercules, JRS and Ronas Chemicals.

A buffer is employed in the present invention to maintain the pH of the pharmaceutical composition in the range of from 5 to 7, preferably from 6 to 7, e.g. pH 6.6. Typical buffers which may be employed in the composition include disodium phosphate dodecahydrate and sodium dihydrogen phosphate dihydrate. The amount of buffer employed should be sufficient to retain the pH of the composition within the specified range. For example, the amount of buffer may be from 0.1 to 1% by wt. Preferably, a phosphate buffer pH 6.6 (USP23) is employed.

In the present invention, the pharmaceutical composition may include a chelating agent, such as the disodium salt of ethylene diamine tetraacetic acid (hereafter EDTA). The amount of chelating agent may be from 0.01 to 10% by wt, preferably 0.1 to 5% by wt, more preferably 0.1 to 1% by wt. The EDTA can also act as a preservative in the pharmaceutical composition.

In the present invention, the pharmaceutical composition may include an isotonicity regulator, such as sodium chloride. The isotonicity regulator may be present in the composition in an amount of from 0.01 to 5% by wt, preferably from 0.1 to 1% by wt.

In the present invention, the pharmaceutical composition may comprise one or more other components, e.g. preservatives, such as benzalkonium chloride, and crystallisation inhibitors, such as sorbitol. However, it is preferred that the composition is free of benzalkonium chloride. It is also preferred that the composition is free of sorbitol. It is even more preferred that the composition is free of both benzalkonium chloride and sorbitol.

According to one particular embodiment, the present invention provides a method for the preparation of an aqueous nasal pharmaceutical composition comprising:
a) 0.005 to 10% by wt, preferably 0.01 to 5% by wt, more preferably 0.25 to 2.5 wt %, most preferably 0.25 to 1.5% by wt of one or more water-soluble pharmaceutically active ingredients suitable for nasal administration;
b) from 0.01 to 10% by wt, preferably from 0.05 to 5% by wt, more preferably 0.1 to 5% by wt, most preferably 0.1 to 2% by wt, hydroxypropyl methyl cellulose having a viscosity of from 2500 to 5500 cps (mPa·s), preferably more than 3000 to less than 5000 cps (mPa·s), more preferably from 3200 to 4800 cps (mPa·s), and
c) a buffer for maintaining the pH of the aqueous pharmaceutical composition at from 5 to 7,
   wherein the method comprises:
i) dissolving the above components in water to form an aqueous solution, and
ii) filtering the aqueous solution formed in i) through a sieve to form an aqueous nasal pharmaceutical composition;
   characterised in the that the sieve through which the aqueous solution is filtered has a mesh size from ≧1 micron but ≦10 microns, preferably from 1.5 to 5 microns, most preferably 2 to 4 microns, e.g. 3 microns.

In this particular embodiment, the water soluble pharmaceutically active ingredient is preferably selected from vasoconstrictors, antiallergic agents, antiemetics, bronchodilators, antiseptics, anesthetics, cytostatics and corticosteroids. The water-soluble vasoconstrictor is preferably selected from xylometazoline e.g. xylometazoline hydrochloride, xylometazoline, indanazoline, metizoline, naphazoline e.g. naphazoline hydrochloride, fenoxazoline e.g. fenoxazoline hydrochloride, oxymetazoline e.g. oxymetazoline hydrochloride, tetrahydrozoline, tramazoline, tymazoline, phenylephrine e.g. phenylephrine hydrochloride, ephedrine e.g. d-pseudoephedrine hydrochloride, or epinephrine. The water soluble vasoconstrictor is most preferably xylometazoline e.g. xylometazoline hydrochloride. The water soluble antiallergic agent is preferably selected from (1) cromoglycic acid or a nasally acceptable salt thereof, e.g. disodium cromoglycate), or (2) H1 receptor antagonists, such as dimethindene or a nasally acceptable salt thereof, e.g. dimethindene maleate, acrivastine, brompheniramine, chlorpheniramine, dexchlorpheniramine, triprolidine, bromodiphenhydramine, clemastine, phenyltoloxamine, piprinhydrinate, pyrilamine, tripelennamine, azelastine, cetirizine, levocetirizine, hydroxyzine, methdilazine, promethazine, trimeprazine, azatadine, cyproheptadine, loratadine, desloratadine, astemizole, diphenhydramine, levocabastine orterfenadine. The water soluble corticosteroid is preferably selected from beclomethasone e.g. beclomethasone dipropionate, and fluticasone e.g. fluticasone propionate. The water-soluble antiemetic is preferably metoclopramide e.g. metoclopramide hydrochloride. The active ingredient may comprise a mixture of more than one water soluble active ingredient.

The aqueous nasal pharmaceutical composition may include disodium phosphate dodecahydrate and/or sodium dihydrogen phosphate dihydrate as a buffering agent.

The aqueous nasal pharmaceutical composition may include a chelating agent, such as the disodium salt of ethylene diamine tetraacetic acid.

The aqueous nasal pharmaceutical composition may include an isotonicity regulator, such as sodium chloride.

The aqueous nasal pharmaceutical composition is preferably free of a preservative which is not also a chelating agent.

The composition may also contain other ingredients commonly found in nasal pharmaceutical compositions.

In the present invention, the aqueous nasal pharmaceutical composition is preferably provided in a form such that it can be administered as one or more drops or as a spray.

In the present invention, the composition to be filtered can be prepared as a solution using conventional mixing and dissolution techniques. The solution is then filtered through a sieve with a mesh of the above specified size using conventional filtering techniques.

The invention shall now be illustrated by way of the following worked examples and with reference to the drawing, which examples are not intended to be limiting upon the scope of the invention.

FIG. 1 is a view of apparatus used in Example 8.

### EXAMPLE 1

### Method for Preparation of Pharmaceutical Composition Comprising 10 mg/g Xylometazoline Hydrochloride

A pharmaceutical composition having the formulation set out in Table 1 below was prepared substantially in accordance with the disclosure of Example 2 of WO-A-03070213.

| Components | % by wt |
|---|---|
| HPMC (Grade 4000 cps (mPa · s)) | 0.75 |
| Na₂ EDTA | 0.5 |
| Xylometazoline hydrochloride | 0.1 |
| NaCl | 0.38 |
| Phosphate Buffer pH 6.6 (US Pharmacopoeia 23) | Balance to 100 |

Accordingly, to 98.27 parts by wt. of an aqueous phosphate buffer solution with a pH 6.6 at 20° C., is added 0.38 parts by wt NaCl, continuously mixing with a magnetic mixer (1000 r.p.m.) for 10 min. After that the solution is heated to 50° C. Then 0.75 parts by wt HPMC (grade 4000 cps) is mixed with 0.50 parts by wt EDTA-disodium salt. The mixture is added in small quantities to the 15 parts of the buffer solution, continuously mixing at 1000 r.p.m. The mixing is continued for 10 min. at 50° C., and then gradually cooled to 20° C. over the next 3 hours. The obtained gel is left for 24 hours at 18-20° C. The gel is diluted by addition of 68.65 parts by wt. buffer at 20° C. under continuous stirring at 1000 r.p.m. and then continuously stirred for a further 30 min. to provide Solution A.

Xylometazoline hydrochloride (0.1 parts by wt) is dissolved in 15 parts by wt of the phosphate buffer at 20° C. by continuous stirring at 1000 r.p.m. and then continuously stirred for a further 10 min. to provide Solution B.

Solution B is added to Solution A at 20° C. while continuously stirring at 1000 r.p.m. and then continuously stirred for a further 10 min. The resultant solution from the mixing of Solution A and Solution B is complemented to 100 parts by weight of distilled water, if necessary, and the bubbles removed with stirring (500 r.p.m.) under lowered pressure to provide Solution C.

Solution C is then filtered through a 3 μm sieve to provide a pharmaceutical composition of the present invention.

### EXAMPLES 2-7 AND 2′-7′

### Method for Preparation of Pharmaceutical Composition Comprising 2, 3, 6, 8, 10 and 15 mg/g HPMC

Examples 2-7 and 2′-7′ were prepared according to the method set out above for Example 1, except that the amount of HPMC was varied to provide pharmaceutical compositions having the specified level of HPMC. Further, in Examples 2-7 Solution C was filtered through a 3 micron sieve, whereas in Examples 2′-7′ Solution C was not filtered.

### EXAMPLE 8

### Method for Examination of the Relative Adhesive Capacity of Pharmaceutical Compositions 2-7 and 2′-7′

The relative adhesive capacity of the pharmaceutical compositions prepared in each of Example 2-7 and 2′-7′ was determined by the testing procedure for solutions set out in Example 8 of WO-A-03070213. Using the apparatus described in Example 8 and illustrated in FIG. 1, the testing procedure was repeated except that the polymer solutions evaluated in WO-A-03070213 were replaced by the filtered or unfiltered pharmaceutical solutions of Examples 2-7 and 2′-7′.

The apparatus used in the evaluation comprises a microbalance 1 including raising/lowering screw 2, calibration screw 3, and a microforce balance 4.

In summary, and with reference to FIG. 1 hereof, the pharmaceutical solution which is going to be tested is placed into a 15 ml glass vial 6, maintained at a constant temperature by water bath 7. A glass plate 5 is covered with a gelatin film, which acts as a reference polymer imitating the mucus in the nasal cavity. The pharmaceutical solutions are tested for adhesion. The plate (18×18 mm) 5 is suspended from a microforce balance 4 (WAGA TORSJJNA-WT, Techniport, Poland). Using screw 2, the plate 5 is immersed in the 15 ml glass vial 6 filled with the test solution. After a defined time for contact (5 min), the plate is raised gradually, using screw 2. The force required for detachment of the plate from the solution is measured using device 4 and shown on scale 8. The maximum force required to draw the plate out of the solution is considered to relate to the adhesive force between the pharmaceutical solution and the mucus. As a standard, the clean plate is tested before and after coating with the polymer. The force for detachment of the coated plate is expressed as a percentage of the clean plate detachment force, i.e. the results have relative values.

The polymer film was prepared by dropping 0.5 ml of a 1% solution of gelatin and spreading it over the whole plate surface. After a 24 h period (at 20-22 C), necessary for forming a smooth film on a leveled surface, the film was oven-dried at 40° C. to a constant weight. The films obtained were uniform and exhibited a good reproducibility of their weight (sd<5%).

The tests were carried out at 20, 25, 30 and 37° C.

The results of the tests are shown in Table 2.

Relative adhesive capacity (%) of HPMC 4000 solutions (mean ± sd)

| | | | | |
|---|---|---|---|---|
| Example No/ | | | | |
| Concentration | | | | |
| (mg/g) | 20° C. | 25° C. | 30° C. | 37° C. |

HPMC 4000 through 3 μm filter

| | | | | |
|---|---|---|---|---|
| 2/2 | 104.1 ± 3.03 | 118.9 ± 6.23 | 93.5 ± 1.4 | 87.8 ± 1.93 |
| 3/3 | 111.9 ± 4.61 | 122.7 ± 4.24 | 105.7 ± 2.6 | 101.1 ± 2.83 |
| 4/6 | 114.3 ± 3.53 | 125.0 ± 7.07 | 108.2 ± 4.24 | 101.2 ± 2.73 |
| 5/8 | 113.6 ± 3.21 | 123.2 ± 4.69 | 107.0 ± 2.83 | 100.7 ± 1.41 |
| 6/10 | 111.4 ± 2.93 | 120.4 ± 4.16 | 102.0 ± 3.21 | 93.2 ± 2.28 |
| 7/15 | 139.4 ± 4.95 | 128.2 ± 4.24 | 97.3 ± 1.67 | 82.2 ± 1.68 |

HPMC 4000 without filtering

| | | | | |
|---|---|---|---|---|
| 2′/2 | 98.2 ± 4.04 | 113.5 ± 8.69 | 88.3 ± 3.14 | 87.5 ± 3.78 |
| 3′/3 | 100.7 ± 6.95 | 109.2 ± 6.12 | 95.1 ± 4.97 | 98.1 ± 4.19 |
| 4′/6 | 107.5 ± 5.9 | 117.0 ± 8.55 | 93.7 ± 5.12 | 98.4 ± 4.619 |
| 5′/8 | 106.5 ± 6.13 | 116.0 ± 6.18 | 98.1 ± 6.46 | 94.7 ± 4.31 |
| 6′/10 | 107.2 ± 5.81 | 115.0 ± 7.32 | 99.0 ± 5.44 | 84.0 ± 5.2 |
| 7′/15 | 111.1 ± 7.47 | 108.4 ± 8.93 | 95.2 ± 5.96 | 70.2 ± 5.83 |

The results of Table 2 illustrate that filtering the solution in accordance with the present invention (Examples 2-7) provides pharmaceutical solutions having consistent relative adhesive capacities. These improved results will be found by filtering solutions through sieves up to 10 micron. Filtering the solutions through sieves above 10 μm does not cause any improvement in consistency in the reproducibility of relative adhesive capacities.

Further, it will be noted that the compositions of Examples 2-7 will retain their relative adhesive capacities for longer than the unfiltered compositions 2′-7′, when left to stand without stirring at 20° C. for up to 18 months.

1. A method for the preparation of an aqueous pharmaceutical composition comprising:
a) 0.005 to 10% by wt of one or more water-soluble pharmaceutically active ingredients or pharmaceutically acceptable salts thereof;
b) from 0.01 to 10% by wt hydroxypropyl methyl cellulose, having a viscosity of from 2500 to 5500 cps (mPa·s); and
c) a buffer for maintaining the pH of the aqueous pharmaceutical composition from 5 to 7;
wherein the method comprises:
i) dissolving the above components in water to form an aqueous solution; and
ii) filtering the aqueous solution formed in i) through a sieve to form an aqueous pharmaceutical composition;
characterised in that:
the sieve through which the aqueous solution is filtered has a mesh size from ≧1 micron but ≦10 microns.

2. A method as claimed in claim 1, wherein the one or more water-soluble pharmaceutically active ingredients or pharmaceutically acceptable salts thereof is selected from vascoconstrictors, antiallergic agents, antiemetics, bronchodilators, antiseptics, local anesthetics, cytostatics, analgesics (narcotic and non-narcotic), steroidal and non-steroidal anti-inflammatories, topical antibiotics, antiparasitics, antibacterials, anticonvulsants, antispasmodics and anticholinergics, antifungals, antivirals, antidiabetics, antimigraines, hormones, sedatives, antianaphylactics, beta-adrenoceptor agonists, diagnostic drugs, and vaccines.

3. A method as claimed in claim 1, wherein the pharmaceutically active ingredient is a water-soluble vasoconstrictor selected from xylometazoline, indanazoline, metizoline, naphazoline, fenoxazoline, oxymetazoline, tetrahydrozoline, tramazoline, tymazoline, phenylephrine, ephedrine and epinephrine.

4. A method as claimed in claim 1, wherein the pharmaceutically active ingredient is a water-soluble antiallergic agent selected from (1) cromoglycic acid, or (2) H1 receptor antagonists.

5. A method as claimed in claim 1, wherein the pharmaceutically active ingredient is a water-soluble steroidal or non-steroidal anti-inflammatory selected from corticosteroids, diclofenac and celecoxib.

6. A method as claimed in claim 1, wherein the pharmaceutically active ingredient is a water-soluble antiemetic selected from metoclopramide, ondansetron, granisetron, dronabinol, prochloperazine and chlorpromazine.

7. A method as claimed in claim 1, wherein the pharmaceutically active ingredient is a water-soluble narcotic and non-narcotic analgesic selected from morphine, hydromorphine, pentazocine, and acetaminophen.

8. A method as claimed in claim 1, wherein the pharmaceutically active ingredient is a water-soluble anesthetic selected from lidocaine, pramoxine and benzocaine.

9. A method as claimed in claim 1, wherein the pharmaceutically active ingredient is a water-soluble topical antibiotic selected from neomycin and bacitracin.

10. A method as claimed in claim 1, wherein the pharmaceutically active ingredient is a water-soluble antiparasitic selected from metronidazole and quinolines.

11. A method as claimed in claim 1, wherein the pharmaceutically active ingredient is a water-soluble antibacterial selected from tetracycline, erythromycin, quinolone antibacterials, and azithromycin.

12. A method as claimed in claim 1, wherein the pharmaceutically active ingredient is a water-soluble anticonvulsant selected from phenyloin, gabapentin, phenobarbital and carbamazepine.

13. A method as claimed in claim 1, wherein the pharmaceutically active ingredient is a water-soluble antispasmodic or anticholinergic selected from atropine and scopolamine.

14. A method as claimed in claim 1, wherein the pharmaceutically active ingredient is a water-soluble antifungal selected from miconazole, econazole and terconazole.

15. A method as claimed in claim 1, wherein the pharmaceutically active ingredient is a water-soluble antiviral include acyclovir and behenyl alcohol.

16. A method as claimed in claim 1, wherein the pharmaceutically active ingredient is a water-soluble antidiabetic selected from glipizide and glyburide.

17. A method as claimed in claim 1, wherein the pharmaceutically active ingredient is a water-soluble antimigraine selected from sumatriptan and ergotamine.

18. A method as claimed in claim 1, wherein the pharmaceutically active ingredient is a water-soluble hormones selected from insulin, steroidal hormones, calcitonin, melatonin and tissue growth factors.

19. A method as claimed in claim 1, wherein the pharmaceutically active ingredient is a water-soluble sedatives selected from barbiturates and benzodiazepines.

20. A method as claimed in claim 1, wherein the pharmaceutically active ingredient is a water-soluble antianaphylactic selected from adrenaline and epinephrine.

21. A method as claimed in claim 1, wherein the pharmaceutically active ingredient is a water-soluble beta-adrenoceptor agonists selected from ephedrine hydrochloride, salbutamol/albuterol, fenoterol, clenbuterol, salmeterol and formoterol.

22. A method as claimed in claim 1, wherein the pharmaceutically active ingredient is a water-soluble diagnostic drug selected from phenolsulfonphthalein, Dye T-1824, vital dyes, potassium ferrocyanide, secretin, pentagastrin and cerulean.

23. A method as claimed in claim 1, wherein the pharmaceutically active ingredient is a water-soluble vaccine.

24. A method as claimed in claim 1, wherein the water-soluble pharmaceutically active ingredient is selected from one or more of xylometazoline, indanazoline, metizoline, naphazoline, fenoxazoline, oxymetazoline, tetrahydrozoline, tramazoline, tymazoline, phenylephrine, ephedrine, epinephrine, cromoglycic acid, dimethindene, acrivastine, azelastine, brompheniramine, chlorpheniramine, dexchlorpheniramine, triprolidine, bromodiphenhydramine, clemastine, phenyltoloxamine, piprinhydrinate, pyrilamine, tripelennamine, cetirizine, levocetirizine, hydroxyzine, methdilazine, promethazine, trimeprazine, azatadine, cyproheptadine, loratadine, desloratadine, astemizole, diphenhydramine, levocabastine orterfenadine, beclomethasone, fluticasone, diclofenac, celecoxib, metoclopramide, ondansetron, granisetron, dronabinol, prochloperazine, chlorpromazine, morphine, hydromorphine, pentazocine, acetaminophen, lidocaine, pramoxine, benzocaine, neomycin, bacitracin, metronidazole, quinolines, tetracycline, erythromycin, quinolone antibacterials, azithromycin, phenyloin, gabapentin, phenobarbital, carbamazepine, atropine, scopolamine, miconazole, econazole, terconazole, acyclovir, behenyl alcohol, glipizide, glyburide, sumatriptan, ergotamine, insulin, steroidal harmones, calcitonin, melatonin, tissue growth factors, barbiturates, benzodiazepines, adrenaline, epinephrine, salbutamol/albuterol, fenoterol, clenbuterol, salmeterol, formoterol, phenolsulfonphthalein, Dye T-1824, vital dyes, potassium ferrocyanide, secretin, pentagastrin, cerulean, allergens for immunotherapy and oral bacterial vaccines used as immunomodulators, or a pharmaceutically acceptable salt thereof.

25. A method for the preparation of an aqueous nasal pharmaceutical composition comprising:
a) 0.005 to 10% by wt, preferably 0.01 to 5% by wt, more preferably 0.25 to 2.5 wt %, most preferably 0.25 to 1.5% by wt of one or more water-soluble pharmaceutically active ingredients suitable for nasal administration;
b) from 0.01 to 10% by wt, preferably from 0.05 to 5% by wt, more preferably 0.1 to 5% by wt, most preferably 0.1 to 2% by wt, hydroxypropyl methyl cellulose having a viscosity of from 2500 to 5500 cps (mPa·s), preferably more than 3000 to less than 5000 cps (mPa·s), more preferably from 3200 to 4800 cps (mPa·s), and
c) a buffer for maintaining the pH of the aqueous pharmaceutical composition at from 5 to 7,
wherein the method comprises:
i) dissolving the above components in water to form an aqueous solution, and
ii) filtering the aqueous solution formed in i) through a sieve to form an aqueous nasal pharmaceutical composition;
characterised in the that the sieve through which the aqueous solution is filtered has a mesh size from ≧1 micron but ≦10 microns, preferably from 1.5 to 5 microns, most preferably 2 to 4 microns, e.g. 3 microns.

26. A method as claimed in claim 25, wherein the water soluble pharmaceutically active ingredient is selected from vascoconstrictors, antiallergic agents, antiemetics, bronchodilators, antiseptics, anesthetics, cytostatics and corticosteroids.

27. A method as claimed in claim 26, wherein the water-soluble vasoconstrictor is selected from xylometazoline e.g. xylometazoline hydrochloride, indanazoline, metizoline, naphazoline e.g. naphazoline hydrochloride, fenoxazoline e.g. fenoxazoline hydrochloride, oxymetazoline e.g. oxymetazoline hydrochloride, tetrahydrozoline, tramazoline, tymazoline, phenylephrine e.g. phenylephrine hydrochloride, ephedrine e.g. d-pseudoephedrine hydrochloride, or epinephrine.

28. A method as claimed in claim 25, wherein the water-soluble active ingredient is selected from xylometazoline, e.g. xylometazoline hydrochloride, oxymetazoline, e.g. oxymetazoline hydrochloride, and metoclopramide, e.g. metoclopramide hydrochloride.

29. A method as claimed in claim 28, wherein the water soluble active ingredient is xylometazoline e.g. xylometazoline hydrochloride.

30. A method as claimed in claim 26, wherein the water soluble antiallergic agent is selected from (1) cromoglycic acid or a nasally acceptable salt thereof, e.g. disodium cromoglycate), or (2) H1 receptor antagonists, such as dimethindene or a nasally acceptable salt thereof, e.g. dimethindene maleate, acrivastine, brompheniramine, chlorpheniramine, dexchlorpheniramine, triprolidine, bromodiphenhydramine, clemastine, phenyltoloxamine, piprinhydrinate, pyrilamine, tripelennamine, azelastine, cetirizine, levocetirizine, hydroxyzine, methdilazine, promethazine, trimeprazine, azatadine, cyproheptadine, loratadine, desloratadine, astemizole, diphenhydramine, levocabastine orterfenadine.

31. A method as claimed in claim 26, wherein the water soluble corticosteroid is selected from beclomethasone e.g. beclomethasone dipropionate, and fluticasone e.g. fluticasone propionate.

32. A method as claimed in claim 26, wherein the water-soluble antiemetic is metoclopramide e.g. metoclopramide hydrochloride

33. A method as claimed in claim 1, wherein the active ingredient comprises a mixture of more than one water soluble active ingredient.

34. A method as claimed in claim 1, wherein the pharmaceutical composition includes disodium phosphate dodecahydrate and/or sodium dihydrogen phosphate dihydrate.

35. A method as claimed in claim 1, wherein the pharmaceutical composition includes a chelating agent, such as the disodium salt of ethylene diamine tetraacetic acid.

36. A method as claimed in claim 1, wherein the pharmaceutical composition includes an isotonicity regulator, such as sodium chloride.

37. A method as claimed in claim 1, wherein the pharmaceutical composition is free of a preservative which is not also a chelating agent.

38. A method as claimed in claim 1, wherein the hydroxypropyl methyl cellulose has a viscosity of from 2500 to 5500 cps (mPa·s), preferably more than 3000 to less than 5000 cps (mPa·s), more preferably from 3200 to 4800 cps (mPa·s).

39. An aqueous pharmaceutical composition comprising:
a) 0.005 to 10% by wt, preferably 0.01 to 5% by wt, more preferably 0.25 to 2.5 wt %, most preferably 0.25 to 1.5% by wt of one or more water-soluble pharmaceutically active ingredients or pharmaceutically acceptable salt thereof;
b) from 0.01 to 10% by wt, preferably from 0.05 to 5% by wt, more preferably 0.1 to 5% by wt, most preferably 0.1 to 2% by wt, hydroxypropyl cellulose having a viscosity of from 2500 to 5500 cps (mPa·s), preferably more than 3000 to less than 5000 cps (mPa·s), more preferably from 3200 to 4800 cps (mPa·s), and
c) a buffer for maintaining the pH of the aqueous pharmaceutical composition at from 5 to 7,
wherein the composition is obtainable or obtained by the method claimed in any one of the preceding claims.

40. A method as claimed in claim 25, wherein the active ingredient comprises a mixture of more than one water soluble active ingredient.

41. A method as claimed in claim 25, wherein the pharmaceutical composition includes disodium phosphate dodecahydrate and/or sodium dihydrogen phosphate dihydrate.

42. A method as claimed in claim 25, wherein the pharmaceutical composition includes a chelating agent, such as the disodium salt of ethylene diamine tetraacetic acid.

43. A method as claimed in claim 25, wherein the pharmaceutical composition includes an isotonicity regulator, such as sodium chloride.

44. A method as claimed in claim 25, wherein the pharmaceutical composition is free of a preservative which is not also a chelating agent.

45. A method as claimed in claim 25, wherein the hydroxypropyl methyl cellulose has a viscosity of from 2500 to 5500 cps (mPa·s), preferably more than 3000 to less than 5000 cps (mPa·s), more preferably from 3200 to 4800 cps (mPa·s).
